(19) United States
(12) Patent Application Publication
(10) Pub. No.: US 2026/0345732 A1
(43) Pub. Date: Nov. 5, 2026

(54) IL-17 BASED THERAPY

(71) Applicant: UNIVERSITÄTSKLINIKUM ESSEN, Essen (DE)

(72) Inventors: Alexander RÖSCH, Ratlingen (DE); Lisa ZIMMER, Essen (DE); Renata VARALJAI, Essen (DE)

(21) Appl. No.: 19/488,256

(22) PCT Filed: Jun. 24, 2024
(86) PCT No.: PCT/EP2024/067602

§ 371 Date: Nov. 26, 2025

(30) Foreign Application Priority Data
Jun. 30, 2023 (DE) 10 2023 117 283.2

(51) Int. Cl. A61K 38/20; A61K 39/00; A61K 39/395; A61P 35/00; C07K 16/28; G01N 33/5751

(52) U.S. Cl. CPC A61K 38/20; A61K 39/3955; A61P 35/00; C07K 16/2818; G01N 33/5751; A61K 2039/507; A61K 2039/54; A61K 2039/545; G01N 2333/54

(57) ABSTRACT

In a first aspect, the present invention relates to a method of determining whether a subject who has been diagnosed with melanoma is amenable to a treatment with immune checkpoint inhibitor(s) ICI(s) wherein the method comprises determination of the level and/or amount of IL-17 in a biological sample obtained from the subject and drawing a conclusion as to the amenability of said subject to a treatment with ICI(s) from the level and/or amount of IL-17 determined in said biological sample. Moreover, the present invention relates to a method for determining the therapy regimen of a subject who has been diagnosed with melanoma whereby the therapy comprise a treatment with ICI(s). Said method includes the measurement of the level and/or amount of IL-17 in a biological sample obtained from the subject and concluding whether the therapy regimen of said subject is amenable to a treatment with ICI(s) from the determined level or amount of IL-17. Moreover, a method for determining responsiveness of the subject has been diagnosed with melanoma to a treatment with ICI(s) is disclosed. In a further aspect, the present invention relates to the use of IL-17 for predicting response to ICI therapy and/or for stratification of ICI in a subject who has been diagnosed with melanoma, in particular, metastatic melanoma. Moreover, the present invention relates to a pharmaceutical composition comprising a combination of IL-17 with at least one the ICI, in particular, being selected from anti-PD1 ICI and anti-CTLA-4 ICI.

In a first aspect, the present invention relates to a method of determining whether a subject who has been diagnosed with melanoma is amenable to a treatment with immune checkpoint inhibitor(s) ICI(s) wherein the method comprises determination of the level and/or amount of IL-17 in a biological sample obtained from the subject and drawing a conclusion as to the amenability of said subject to a treatment with ICI(s) from the level and/or amount of IL-17 determined in said biological sample. Moreover, the present invention relates to a method for determining the therapy regimen of a subject who has been diagnosed with melanoma whereby the therapy comprise a treatment with ICI(s). Said method includes the measurement of the level and/or amount of IL-17 in a biological sample obtained from the subject and concluding whether the therapy regimen of said subject is amenable to a treatment with ICI(s) from the determined level or amount of IL-17. Moreover, a method for determining responsiveness of the subject has been diagnosed with melanoma to a treatment with ICI(s) is disclosed. In a further aspect, the present invention relates to the use of IL-17 for predicting response to ICI therapy and/or for stratification of ICI in a subject who has been diagnosed with melanoma, in particular, metastatic melanoma. Moreover, the present invention relates to a pharmaceutical composition comprising a combination of IL-17 with at least one the ICI, in particular, being selected from anti-PD1 ICI and anti-CTLA-4 ICI.

### PRIOR ART

Treatment with ICI has become a major pillar for therapy of metastatic melanoma. It is described that blocking antibodies against cytotoxic T-lymphocyte-associated protein 4 (CTLA-4) and programmed cell death protein 1 (PD-1) have shown clinical success and extended the survival of patients with melanoma, see e. g. Huang, A. C., Zappasodi, R., 2022, Nat. Immunol., 23, 660-670. Unfortunately, not all patients benefit to the same extent due to intrinsic mechanisms of tumor immune resistance or resistance that is acquired after an initial response to treatment, actually, the majority relapses or experiences severe immune-related adverse events (irAEs). Hence, it remains a challenge to identify patients who will benefit from treatment in the long term. There is still a lack of feasible biomarkers and mechanistic understanding for risk stratification of melanoma patients prior to ICI therapy, and these lacks needs to be addressed. For example, dual ICI (anti-CTLA-4 in combination with anti-PD1) leads to higher tumor control, but also to more severe immune related adverse events than mono ICI like anti-PD1, see e.g., Wolchok, J. D., et al., 2022, J Clin Oncol 40, 127-137.

Recently, unexpected observations were reported from the Checkmade 067 and IMMUNED trials identifying that patients harbouring the BRAF V600 mutation (BRAF mt) had longer survival than patients with wild type BRAF (BRAF wt) when treated with the combination of anti-CTLA-4 ICI and PD-1 ICI. This is different to the observation with monotherapy of each of the ICI showing only small survival differences when stratified according to BRAF mutations.

Prior art described various biomarker and treatment combination which may influence the survival rate and relapse of tumor affected individuals. As said, due to lack of physical biomarkers and mechanistic understanding, the risk stratification prior to the therapy ICI therapy, is highly demanded.

The interleukin 17 (IL-17) family includes six structurally relevant members (IL-17 A-F) and is a pro-inflammatory cytokine produced by a subset of CD4+ T-cells, primary Th17 cells, CD8+ T-cells and various innate immune cells types. Prior art discloses, that IL-17 has an essential role in a multitude of autoimmune diseases and inflammation. IL-17A is the hell mark cytokine of Th17 cells and is the most potent induces of downstream cytokines and neutrophil recruitment among IL-17 family members. The Th17 cells as well as IL-17 have been reported to have both anti-tumor and pro-tumor effects. Several reports suggest that particularly inflamed tumors respond better to ICI in the presence of IL-17, it is controversial whether Th17/IL-17-inflammation could have an anti-tumor effect in melanoma, particularly ICI therapy, e.g., see review of Chen, C. & Gao, F. H., Front. Immunol. 10,187 (2019).

In view of the above, there is still a need for suitable biomarkers in particular with respect to stratifying the therapy regimen including ICI therapy in advance. Thus, it is in an object of the present invention to provide suitable biomarker based methods for predicting responsiveness of a subject to a treatment with ICI(s) and determine whether the subject diagnosed with melanoma is amenable to a treatment with ICI(s) accordingly.

### BRIEF DESCRIPTION OF THE PRESENT INVENTION

In a first aspect, the present invention relates to a method of determining whether a subject who has been diagnosed with melanoma is amenable to a treatment with immune checkpoint inhibitors(s) (ICI) (s) wherein the method comprises:
a) Measuring the level and/or the amount of IL-17 in a biological sample obtained from the subject;
b) drawing a conclusion as to the amenability of said subject to a treatment with ICI(s) from the level and/or amount of IL-17 measured in step a).

In a second aspect, the present invention relates to a method of determining the therapy regimen of a subject who has been diagnosed with melanoma whereby the therapy comprises a treatment with ICI(s), wherein the method comprises:
a) Measuring the level and/or the amount of IL-17 in a biological sample obtained from the subject;
b) drawing a conclusion as to the therapy regimen of said subject to a treatment with ICI(s) from the level and/or amount of IL-17 measured in step a).

In a third aspect, the present invention relates to a method of determining responsiveness of a subject who has been diagnosed with melanoma to a treatment with ICI wherein the method comprises
a) Measuring the level and/or the amount of IL-17 in a biological sample obtained from the subject;
b) drawing a conclusion as to the extent of responsiveness of said subject to a treatment with ICI(s) from the level and/or amount of IL-17 measured in step a).

It has been recognized that depending on the IL-17 measured, it is possible to identify in advance of a suitable treatment regimen with ICI.

In a further aspect, the present invention relates to the use of IL-17 for predicting a response to ICI therapy and/or for stratification of ICI therapy a subject who has diagnosed with melanoma, in particular, metastatic melanoma.

Moreover, a pharmaceutical composition comprising a combination of IL-17 with at least one of the ICI selected from anti-PD1 ICI and anti-CTLA4 ICI for use in treating melanoma in a subject who has been diagnosed with melanoma in particular, metastatic melanoma is disclosed. Finally, the use of a kit for
a) Determining whether a subject who has been diagnosed with melanoma is amenable to a treatment with ICI(s), or
b) of determining the therapy regimen of a subject who has been diagnosed with melanoma whereby the therapy comprise the treatment with ICI(s) or
c) determining responsiveness of a subject who has been diagnosed with melanoma to a treatment with ICI(s) wherein the kit comprises: i) means for measuring IL-17 in a biological sample and ii) instructions on how to use the kit in a method according to the present invention is disclosed.

### BRIEF DESCRIPTION OF THE DRAWINGS

FIG. 1. Interleukin-17 pathway genes are associated with improved response to dual immune checkpoint inhibition. (a) Kaplan-Meier plot for OS according to the IL-17 signaling GES (KEGG: hsa04657) in the TCGA-SKCM cohort. (b-g) Kaplan-Meier plots for PFS (b-d) and OS (e-g) according to the IL-17 family GES (‘IL17A-F GES’: IL-17 family cytokines containing the six structurally related cytokines) in dual ICI- (b/e), mono CTLA-4- (c/f), and in mono PD-1-treated patients (d/g). HR and 95% CI are reported for high expression groups. p-values represent log rank test. Categorization into ‘high’ vs. ‘low’ was done according to an optimal cut point. All p-values are two-tailed. TCGA: The Cancer Genome Atlas, SKCM: Skin cutaneous melanoma, GES: gene expression signature, KEGG: Kyoto Encyclopedia of Genes and Genomes, OS: overall survival, PFS: progression-free survival, HR: hazard ratio, CI: confidence intervals.

FIG. 2. IL-17A supports anti-tumor effects of dual ICI. (a) Tumor growth kinetics of transplanted CM (BRAF wt ICI-sensitive) melanoma tumors treated with IgG/H₂O (control, n=6), α-CTLA-4+α-PD-1 (n=6), α-CTLA-4+α-PD-1+rm-IL-17A (n=6), and α-CTLA-4+α-PD-1+α-IL-17A (n=6) according to the depicted treatment schedule. Data points show mean±SEM until the day of first mice eliminated from each group, p-values from 1-way ANOVA with Tukey's multiple comparisons test. (b) Corresponding serum IL-17A levels in mice grouped according to final tumor volume (n=6<800 mm³ vs. n=10≥800 mm³ in size biologically independent samples). Bar plot shows the mean±SEM, and p-value is from unpaired t-test. (c) Delta values of human IFN-v, CXCL10 and CXCL9 secreted by melanoma PDTFs upon either α-CTLA-4+α-PD-1 or α-CTLA-4+α-PD-1+α-IL-17A ex vivo treatment. All p-values are two-tailed. Wt: wild-type, ICI: immune checkpoint inhibition, S: sensitive, s.c.: subcutaneous, ♀: female, rm: recombinant mouse.

FIG. 3. IL-17A/Th17 profiling for response prediction in ICI-treated melanoma patients. (a) Plasma IL-17A levels as measured by ELISA in correlation to best clinical response in samples collected at therapy baseline (BL, n=41 R vs. n=29 NR) and at early follow-up (FU, n=33 R vs. n=12 NR) visits in the dual ICI-treated melanoma patient cohort (first-line α-CTLA-4+α-PD-1, n=70). (b) Corresponding Kaplan-Meier plot for PFS according to the baseline IL-17A concentration in the dual ICI-treated melanoma patient cohort. (c) Plasma IL-17A levels as measured by ELISA in correlation to best clinical response in samples collected at BL (n=19 R vs. n=32 NR) and at early FU (n=11 R vs. n=14 NR) visits in the mono α-PD-1-treated melanoma patient cohort (first-line α-PD-1, n=51). (d) Corresponding Kaplan-Meier plot for PFS according to the baseline IL-17A concentration in the mono α-PD-1-treated melanoma patient cohort. All p-values are two-tailed. p-values are from unpaired t-test with Welch's correction and the mean±95% CI is plotted in panels a/c. Each dot represents a biologically independent sample. HR and 95% CI are reported for ‘IL-17A high’ and p-values are from log rank test in panels b/d. Categorization into ‘high’ vs. ‘low’ was done according to the X-tile determined within each dataset. BL: baseline, FU: follow-up, R: responder (complete and partial response), NR: non-responder (progressive disease, mixed response), interm.: intermediate, HR: hazard ratio, CI: confidence intervals.

### DETAILED DESCRIPTION OF THE PRESENT INVENTION

In a first aspect, the present invention relates to a method of determining whether a subject who has been diagnosed with melanoma is amenable to a treatment with immune checkpoint inhibitors(s) (ICI) (s) wherein the method comprises:
a) Measuring the level and/or the amount of IL-17 in a biological sample obtained from the subject;
b) drawing a conclusion as to the amenability of said subject to a treatment with ICI(s) from the level and/or amount of IL-17 measured in step a).

In a second aspect, the present invention relates to a method of determining the therapy regimen of a subject who has been diagnosed with melanoma whereby the therapy comprises a treatment with ICI(s), wherein the method comprises:
a) Measuring the level and/or the amount of IL-17 in a biological sample obtained from the subject;
b) drawing a conclusion as to the therapy regimen of said subject to a treatment with ICI(s) from the level and/or amount of IL-17 measured in step a).

In a third aspect, the present invention relates to a method of determining responsiveness of a subject who has been diagnosed with melanoma to a treatment with ICI wherein the method comprises
a) Measuring the level and/or the amount of IL-17 in a biological sample obtained from the subject;
b) drawing a conclusion as to the extent of responsiveness of said subject to a treatment with ICI(s) from the level and/or amount of IL-17 measured in step a).

As used herein, the term “amenable to a treatment with ICI(s)” refers to a therapy or treatment with active agents falling under the definition of an immune checkpoint inhibitor. That is, beneficial effects in the treatment of individuals or subjects diagnosed with melanoma are described for the active agent.

The term “immune checkpoint inhibitor” refers to active agents suitable in checkpoint inhibitor therapy which is a form of cancer immunotherapy. Immune checkpoints are regulators of the immune systems and typically immune checkpoint inhibitors (ICI) allow potential use in multiple types of cancers. That is, drugs or drug candidates that inhibit/block the inhibitory checkpoint molecules are referred to as ICI(s). Presently, the members of checkpoint inhibitors as described in the art and already approved checkpoint inhibitors target the molecules CTLA-4, PD-1, and PD-L1, respectively. Other inhibitory checkpoint molecules include A2AR and A2BR, B7-H3, B7-H4, BTLA, IDO, KIR, LAG3, NOX2, TIM3, VISTA, SIGLEC7 and SIGLEC9. Further, CISH (cytokine-inducible SH2-containing proteins) have been described as suitable checkpoint inhibitors.

Approved immune checkpoint inhibitors include drugs targeting CTLA-4, like Ipilimumab, Tremelimumab as well as drugs targeting PD-1, like Nivolumab, Pembrolizumab and Cemiplimab. With respect to the PD-L1 target, Atezolizumab, Avelumab and Durvalumab have been described.

With respect to immune checkpoint inhibitors, several adverse-events have been described for example severe immune-related adverse events (irAEs) such as but not limited to colitis, hepatitis, pneumonitis, pancreatitis, dermatitis, thyroiditis, myositis, carditis, hypophysitis which are the most common to occur.

As used herein, the term “therapy regimen” refers to a regimen defining the drugs to be used, their dosage, the frequency and duration of treatments and other considerations. The therapy regimen is the output of therapeutic decision making and patient stratification upfront to treatment with suitable active agents.

It is desired to allow determination of therapy regimen prior to the treatment start, however, it is sometimes required to adjust the therapy treatment due to effects observed during therapy.

The terms “therapy” and “treatment” are used herein inter-changeably unless otherwise indicated.

The therapy is typically a therapeutic therapy; however, preventive therapy may be envisaged.

At present, suitable biomarkers for responsive prediction and patient stratification in melanoma, in particular, when considering ICI treatment, are not described in the art. With respect to the term “responsiveness of a subject . . . of a treatment with ICI” the beneficial effect of the treatment to said subject is considered. In particular, the level of responsiveness is determined. For example, the responsiveness may allow to determine whether a monotherapy is sufficient or whether a combination of two active agents, in particular, ICI is required.

As used herein, the terms “individual” and “subject” are used into changeably unless otherwise indicated.

The term “IL-17” as used herein refer to a member of the interleukin 17 family including the six structurally relevant members of IL-17A, IL-17B, IL-17C, IL-17D, IL-17E, and IL-17F.

The terms “increased” and “elevated” are used interchangeability herein unless otherwise indicated. In particular, an “increased level or amount” or “elevated level or amount” corresponds to an increase by at least 20% of the level or amount compared to the average level or amount of a total population of melanoma patients examined.

It has been recognized that IL-17 and TH17 cells in general represent suitable biomarkers associated with responses to ICI. In addition, it has been identified that combining IL-17 administration with at least one ICI is beneficial in the treatment of melanoma accordingly. The inventors determined that plasma IL-17/TH17 cytokines represent a valuable biomarker at therapy baseline (BL), namely, upfront to the treatment with the ICI response prediction and patient stratification in melanoma, specifically to predict a potential benefit of adding anti-CTLA4 to anti-PD-1 upfront to therapy.

IL-17 determined in a biological sample obtained from the subject, typically, the sample is provided in vitro for measurement of IL-17, represents a suitable biomarker identifying patients where a combinatorial therapy with ICI(s) in particular, anti-CTLA-4 ICI and anti-PD-1 ICI is beneficial as it is the use for TH17 gene expression signatures (GES) in general. That is, in an embodiment of the present invention, the ICI comprises at least one of an anti-PD-1 ICI and/or an anti-CTLA-4 ICI. In an embodiment, a combination thereof is administered.

Further, according to an embodiment of the present invention the IL-7 measured is IL-7A.

The measurement in the biological sample is conducted by known means. The biological sample may be a tissue or a liquid obtained from the subject. In an embodiment, the biological sample is blood, like plasma or serum or whole blood.

Typically, IL-17 level and/or amount is determined on protein level.

Further, the melanoma may be any type of melanoma, like cutaneous melanoma, ocular melanoma, mucosal melanoma, melanoma of unknown primary and any stage of disease from primary tumor to local or systemic metastatic stage. In an embodiment, the melanoma is a metastatic melanoma of stages III and/or IV.

Further, the method according to the present invention is a method wherein elevated levels of IL-17 measured in step a) is indicative for beneficial treatment with a combinatorial therapy of at least two different ICI(s), in particular, treatment with a combination of anti-PD-1 ICI and anti-CTLA-4 ICI, whereas when the IL-17 level or amount is not increased, monotherapy with a single ICI selected from an anti-PD-1 ICI or anti-CTLA-4 ICI is suitable, optionally the treatment or therapy is combined with an administration of IL-17.

Namely, the level and/or amount of IL-17 may be indicative for the combinatory therapy of at least two different ICI(s) or may be indicative for a monotherapy with a single ICI whereby the monotherapy may either be anti-PD-1 ICI or anti-CTLA-4 ICI. In an embodiment, the beneficial treatment may be a treatment wherein the administration of ICI is combined with administration of IL-17. As discussed further below, it has been recognized that IL-17 administered before or during therapy is beneficial in survival rate as well as reduction of relapse of melanoma. Moreover, the administration of IL-17 allows to reduce the dosage of the (ICI(s) or allows to concentrate on monotherapy of ICI, thus, reducing the known adverse effects (irAEs) described in the art.

As demonstrated below, elevated or increased levels or amounts of IL-17, namely, with the existing IL-17 signaling at therapy baseline benefit more from dual ICI therapy. If the IL-17 is not elevated or increased, a monotherapy may be sufficient or a monotherapy combined with IL-17 administration will be suitable. It is considered that the IL-17 pathway supports melanoma response to dual ICI therapy, thus, representing firstly a biomarker for patient stratification and secondly represents a possibility for combined therapy thus reducing adverse effects described for ICI accordingly.

In an embodiment, the method according to the present invention is a method wherein, if i) it is determined that the subject has an increased level and/or amount of IL-17 in the biological sample, typically blood, ii) the subject is diagnosed with melanoma, in particular, metastatic melanoma, and iii) ICI treatment is considered for the therapy of choice and the subject is likely to benefit from treatment with ICI, a therapeutic comprising a combination of two different ICI, in particular, an anti-PD-1 ICI and anti-CTLA-4 ICI, optionally, with IL-17, should be administered to the subject accordingly.

In a further aspect, the present invention relates to the use of IL-17 or TH17 GES in general for prediction a response to ICI therapy and for stratification of ICI therapy of the subject who has been diagnosed with melanoma, in particular, metastatic melanoma. As described above, IL-17 represents a suitable biomarker for patent stratification, in particular, stratifying and determining whether the ICI therapy should be a combinatorial therapy, e.g. of anti-PD-1 ICI and anti-CTLA-4 ICI eventually improving the survival rate of said subject or whether a monotherapy with either anti-PD-1 ICI or anti-CTLA-4 ICI is sufficient.

Moreover, the present invention discloses a pharmaceutical composition comprising a combination of IL-17 with at least one ICI, in particular, with at least one of the ICI(s) selected from anti-PD-1 ICI and anti-CTLA-4 ICI for use in treating melanoma in a subject that has been diagnosed with melanoma. In an embodiment, the subject is a subject being diagnosed with metastatic melanoma. In an embodiment, the pharmaceutical composition according to the present invention comprises a combination of anti-PD-1, anti-CTLA-4 and IL-17 for use in treating metastatic melanoma when an increased level and/or amount of IL-17 is determined in a biological sample, typically provided in vitro, from the subject upfront the therapy.

In some embodiments, the pharmaceutical composition according to the present invention, further comprises at least one pharmaceutically acceptable carrier. In some embodiments, the compounds according to the present invention or a pharmaceutically acceptable salt, solvate or hydrate thereof may be included in a pharmaceutically acceptable carrier.

As used herein and throughout the entire description, the terms “carrier” and “excipient” are used interchangeably herein. Pharmaceutically acceptable carriers or excipients include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO2), solvents/co-solvents (e.g. aqueous vehicle, Propylene glycol, glycerol), buffering agents (e.g. citrate, gluconates, lactates), preservatives (e.g. Na benzoate, para-bens (Me, Pr and Bu), BKC), anti-oxidants (e.g. BHT, BHA, Ascorbic acid), wetting agents (e.g. polysorbates, sorbitan esters), anti-foaming agents (e.g. Simethicone), thickening agents (e.g. methylcellulose or hydroxyethylcellulose), sweetening agents (e.g. sorbitol, saccharin, aspartame, acesulfame), flavoring agents (e.g. peppermint, lemon oils, butterscotch, etc), humectants (e.g. propylene, glycol, glycerol, sorbitol). The person skilled in the art will readily be able to choose suitable pharmaceutically acceptable carriers or excipients, depending, e.g., on the formulation and administration route of the pharmaceutical composition.

A non-exhaustive list of exemplary pharmaceutically acceptable carriers or excipients includes (biodegradable) liposomes; microspheres made of the biodegradable polymer poly(D,L)-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloi-dosomes, niosomes, discomes, proniosomes, microspheres, microemulsions and polymeric micelles. Other suitable pharmaceutically acceptable excipients are inter alia described in Remington's Pharmaceutical Sciences, 15th Ed., Mack Publishing Co., New Jersey (1991) and Bauer et al., Pharmazeutische Technologie, 5th Ed., Govi-Verlag Frankfurt (1997).

The pharmaceutical composition of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration.

Formulations and compositions thus may be designed in accordance with the invention for delivery by any suitable route of administration. In the context of the present invention, the routes of administration include:
enteral routes (such as oral, gastrointestinal, sublingual, sublabial, buccal, rectal); and
parenteral routes (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal).
   In some embodiments the administration may be a parenteral route, in particular intravenous or intramuscular but also intratumoral or peritumoral.

In some embodiments, the pharmaceutical composition, as disclosed herein, is administered to a subject in need thereof in an amount effective to treat cancer. The subject is preferably a human.

As used herein and throughout the entire description, the term “amount effective” in the context of a composition or dosage form for administration to a subject refers to an amount of the composition or dosage form sufficient to provide a benefit in the treatment of cancer, to delay or minimize symptoms associated with cancer, or to cure or ameliorate cancer. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

The administration of the different components of the pharmaceutical compositions may be simultaneously, separately or sequentially.

That is, the administration and therapy regimen may be as follows:
administration of IL-17 i.v. followed by ICI i.v. (mono or combination), with different time intervals between the two infusions (like 1, 2, 3, 4, 5, 6, 7, 14 days), not to be bound by theory, it is considered that IL-17 primes the tumor immune environment, so that subsequent ICI works better, namely more efficient, e.g. at lower dosage, thus reducing adverse effects.
administration of ICI (mono or combination) i.v. followed by IL-17 i.v.
administration of IL-17/ICI(s) in the same i.v. at the same time.
administration of IL-17 intratumorally or peritumorally followed by ICI i.v. (mono or combination), with different time intervals in between infusions (like 1, 2, 3, 4, 5, 6, 7, 14 days).
administration of ICI(s) (mono or combination) i.v. followed by IL-17 peritumorally or intratumorally.
administration of ICI(s) (mono or combination) i.v. and IL-17 peritumorally or intratumorally at the same time.

Finally, the present invention relates to the use of a kit for
a) Determining whether a subject who has been diagnosed with melanoma is amenable to a treatment with ICI(s), or
b) of determining the therapy regimen of a subject who has been diagnosed with melanoma whereby the therapy comprise the treatment with ICI(s) or
c) determining responsiveness of a subject who has been diagnosed with melanoma to a treatment with ICI(s)
wherein the kit comprises: i) means for measuring IL-17 in a biological sample and ii) instructions on how to use the kit in a method.

The present invention will be described further by way of examples without limiting the same.

### Methods

### Analysis of Transcriptomic Datasets

The discovery cohort consisted of pre-treatment tissue samples from 77 treatment naïve BRAF V600E/K mutant melanoma patients from the Combi-v phase 3 study, and 79 treatment naïve BRAF wt patients from the Dermatology Department of the University Hospital Essen. Custom designed CodeSet (containing 780 genes involved in phenotypic resistance) and the commercially available Immune Panel from NanoString™ (800 genes involved in immune pathways) was used to generate expression data on the NanoString™ platform (NanoString Technologies). Clinical parameters of the discovery patient cohort and corresponding gene expression data processing were previously described Brase, J. C. et al., Clinical Cancer Research (2021). https://doi.org:10.1158/1078-0432.ccr-20-3586. The validation cohorts consisted of open-source bulk tumor tissue transcriptomic datasets from The Cancer Genome Atlas Skin Cutaneous Melanoma cohort (TCGA-SKCM, ICI- and MAPKi—naïve melanoma patients) and ICI (Liu, D. et al., Nature Medicine (2019). https://doi.org: 10.1038/s41591-019-0654-5,—phs000452.v3.p1, Van Allen, E. M. et al., Science (2015). https://doi.org:10.1126/science.aad0095—phs000452.v2.p1, Gide, T. N. et al., Cancer Cell (2019). https://doi.org:10.1016/j.ccell.2019.01.003—PRJEB23709, Riaz, N. et al.; Cell (2017). https://doi.org:10.1016/j.cell.2017.09.028—GSE91061) or MAPKi receiving melanoma patients (Long, G. V. et al., Nature Communications (2014). https://doi.org:10.1038/ncomms6694—GSE61992, Rizos et al, Clinical Cancer Research (2014). https://doi.org:10.1158/1078-0432.CCR-13-3122—GSE50509, Kakavand, H. et al. Clinical Cancer Research (2017). https://doi.org:10.1158/1078-0432.CCR-16-1688—GSE99898). Normalized and log₂ transformed RSEM counts (RNA-seq by Expectation Maximization) from the TCGA-SKCM cohort was retrieved from the GDAC Firehose (http://gdac.broadinstitute.org). In the SKCM cohort samples with available mRNA expression and mutation data (n=363) were analyzed. Normalized gene level expression in transcripts per million (TPM) from the Liu et al, see above, RNA-seq dataset was downloaded as provided in the original manuscript. Raw gene expression counts from the Van Allen et al, see above, study was normalized using the DESeq2 method. RNA-seq raw reads from the Gide et al, see above, and Riaz et al, see above, studies were downloaded and converted to TPM using the kallisto method. For Kaplan Meier curves similar treatment arms from ICI datasets were pooled and analyzed for PFS: Liu et al dataset: n=47 α-CTLA-4 [pre-treatment]+α-PD-1, n=74 α-PD-1; Gide et al dataset: n=32 α-CTLA-4+α-PD-1, n=41 α-PD-1 and for OS: Liu et al dataset: n=47 α-CTLA-4 [pre-treatment]+α-PD-1, n=74 α-PD-1; Riaz et al dataset: n=20 α-PD-1; Gide et al dataset: n=32 α-CTLA-4+α-PD-1, n=40 α-PD-1; Van Allen et al: n=42 α-CTLA-4. Categorization into IL-17 A-F GES low vs. high was done separately in each dataset according to optimal cutpoint determined in X-tile, see Camp, R. L., et. al., Clinical Cancer Research 10, 7252-7259 (2004). https://doi.org:10.1158/1078-0432.CCR-04-0713. Raw gene expression profiling data from the MAPKi datasets featuring a uniform, treatment-naïve BRAF V600 mt positive patient cohort by Long et al, Rizos et al, and Kakavand et al were downloaded from the Gene Expression Omnibus. Count matrices were imported in Partek® Flow® where background correction, quantile normalization and log₂ transformation were carried out. In all validation datasets, the IL-17 A-F GES gene family signature consisted of IL-17 family genes with reliable read counts (expression value >0 in at least 60% of tumor samples). Gene expression values were summarised into a single GES score without weighing in the normalized dataset. Immune cell fraction enrichment analyses from RNA-seq datasets were computed according to the Bindea, G. et al., Immunity (2013) immune cell signature using the xCell algorithm, Aran, D., Hu, Z. & Butte, A. J., Genome Biology (2017). https://doi.org:10.1186/s13059-017-1349-1.

### Statistics & Reproducibility

The melanoma patient cohort size calculation for cytokine analyses was based on power analysis using Chi-squared statistic assuming a relative risk of 2.0 between outcome positive and outcome negative proportions (type I/II errors at 0.05 and 0.20, respectively). For in vivo experiments group size was determined based on data from preliminary experiments to detect >20% effect between groups (type I/II errors at 0.05 and 0.20, respectively). In all experiments a minimum of n=4 mice were used to ensure a balance between statistical needs and animal welfare. For all other experiments no sample size calculation was performed, however, reproducibility of the method has been demonstrated on minimum three biologically independent samples. No patients or cohorts were excluded from the analyses. From public datasets only the samples with available baseline gene expression, mutational data and clinical annotation were analyzed. Data collection and analysis were performed blinded for human cytokine analyses. Data were not randomized. Normality distribution was assessed by D'Agostino & Pearson test. Differentially expressed gene set (DEG) analyses were performed using false discovery rate (FDR) applying a two-stage step-up multiple test correction with q≤0.05 cutoff (significant genes are given in Table 1).

TABLE 1
Significantly differentially expressed genes between BRAF
wt vs. BRAF mt tumor tissues in the discovery cohort.

| | | | SE of | | |
|---|---|---|---|---|---|
| Gene name | p - value | Difference | difference | t ratio | q value |
| STAM | <0.000001 | −0.4345 | 0.07654 | 5.676 | 0.000104 |
| ALCAM | 0.001025 | −0.3211 | 0.09592 | 3.348 | 0.049219 |
| CD58 | 0.000043 | −0.2969 | 0.07053 | 4.209 | 0.009664 |
| MAPK1 | 0.000112 | −0.2465 | 0.06218 | 3.964 | 0.017518 |
| RIPK2 | 0.000396 | −0.229 | 0.06322 | 3.622 | 0.039543 |
| YTHDF2 | 0.000021 | −0.2206 | 0.05032 | 4.384 | 0.008175 |
| ABCF1 | 0.000508 | −0.2014 | 0.05669 | 3.552 | 0.039543 |
| PRPF38A | 0.000011 | −0.1993 | 0.04385 | 4.546 | 0.005719 |
| NRAS | 0.000536 | −0.1919 | 0.05425 | 3.536 | 0.039789 |
| MAPK8 | 0.000741 | −0.182 | 0.05287 | 3.443 | 0.043857 |
| TICAM1 | 0.000069 | −0.163 | 0.03983 | 4.092 | 0.013398 |
| EIF2B4 | 0.000505 | −0.1368 | 0.0385 | 3.553 | 0.039543 |
| TFE3 | 0.000459 | 0.169 | 0.04719 | 3.581 | 0.039543 |
| EPHA6 | 0.00089 | 0.2013 | 0.05939 | 3.389 | 0.047846 |
| ORAI2 | 0.00066 | 0.212 | 0.06098 | 3.476 | 0.043857 |
| ELK1 | 0.000026 | 0.2126 | 0.04904 | 4.335 | 0.008175 |
| CXCR1 | 0.000458 | 0.2448 | 0.06837 | 3.581 | 0.039543 |
| HYOU1 | 0.000722 | 0.2529 | 0.0733 | 3.451 | 0.043857 |
| ULBP3 | 0.000394 | 0.2677 | 0.07386 | 3.624 | 0.039543 |
| IFNL1 | 0.00018 | 0.2772 | 0.07221 | 3.84 | 0.023621 |
| ARPC1B | 0.000942 | 0.2901 | 0.086 | 3.373 | 0.048924 |
| IL17A | 0.001068 | 0.3109 | 0.09322 | 3.335 | 0.049219 |
| TEAD2 | 0.000774 | 0.3117 | 0.09085 | 3.43 | 0.043857 |
| INHBA | 0.000788 | 0.3338 | 0.09745 | 3.425 | 0.043857 |
| METTL7B | 0.000725 | 0.3714 | 0.1077 | 3.45 | 0.043857 |
| DMBT1 | 0.000788 | 0.3801 | 0.111 | 3.425 | 0.043857 |
| CNTN6 | 0.000987 | 0.3849 | 0.1146 | 3.359 | 0.049219 |
| PMAIP1 | 0.0001 | 0.399 | 0.09986 | 3.996 | 0.017252 |
| MT3 | 0.000008 | 0.4421 | 0.09558 | 4.625 | 0.005719 |
| FOS | 0.001074 | 0.468 | 0.1404 | 3.334 | 0.049219 |
| CFI | 0.000448 | 0.5693 | 0.1587 | 3.588 | 0.039543 |
| FOSB | 0.000043 | 0.7189 | 0.1707 | 4.212 | 0.009664 |
| ITGB3 | 0.000287 | 0.8011 | 0.2158 | 3.712 | 0.034369 |

p-values are from multiple unpaired t-test (two-tailed) with Benjamini, Krieger and Yekutieli test correction (q).

Gene ontology and pathway enrichment analysis was done on DEGs using FDR (q≤0.05) approach. Statistical significance was calculated using either unpaired t-test or Mann-Whitney U test (depending on normality distribution) in two groups' comparisons, and one-way or two-way ANOVA with multiple comparisons adjustment for more than two groups. Welch's correction was applied under unequal standard deviation (SD) assumption. Categorical data was analyzed by Fisher's exact or chi-squared test. Kaplan Meier plots were computed using survival data categorized according to the biomarker threshold determined using X-tile and curves were compared using the log rank test. GSEA was done using WebGestalt (v.2019), Liao, Y., et. al., Nucleic Acids Research (2019). https://doi.org:10.1093/nar/gkz401, using KEGG, functional database, with FDR≤0.05 significance cutoff. All reported p-values were two-tailed, and p≤0.05 was considered significant. Effect size was estimated according to Hedge's g. Network prediction and pathway enrichment of differentially expressed proteins were carried out in the STRING database 2023. For statistical and bioinformatics data processing GraphPad Prism (v 9.5.1), R studio (R-3.6.1 release) and Partek® Flow® (v10.0.) software were used.

### Cell Culture

Human cell lines: Melanoma cell lines with BRAF V600 mutation (WM983B, 451Lu, WM9) were maintained at 37 °C in a humidified atmosphere with 5% CO₂. Cell lines were obtained from the Wistar Institute (Philadelphia, Pennsylvania) and cultured in 2% FBS-substituted melanoma medium (‘Tu2%’ medium), see Smalley, K. S. M. et al. American Journal of Pathology (2005). https://doi.org:10.1016/S0002-9440 (10)62370-X. 1×10⁵ cells were plated in 6 cm dishes and treated with dabrafenib/trametinib (1 nM/0.2 nM; Selleckchem) or DMSO (0.1%; AppliChem) for 7 days. Media containing drugs were replaced after 3 days.

Murine cell lines: The CM and LN (primary cutaneous melanoma—CM, and lymph node metastasis—LN, derived from ret-transgenic melanoma model, Helfrich, I., et. al., Pigment Cell and Melanoma Research (2014). https://doi.org:10.1111/pcmr.12204), cells were cultured in RPMI medium supplemented with 10% FBS. The YUMM1.7 (ATCC, CRL-3362) and YUMMER1.7 (Merck, SCC243), Wang, J. et al. Pigment Cell and Melanoma Research (2017). https://doi.org:10.1111/pcmr.12591 cells were cultured in DMEM/F-12 supplemented with 10% FBS, 1% NEAA.

Real-Time Quantitative PCR (qPCR)

Total RNA was isolated from cell pellets using the RNeasy Mini Kit according to the manufacturer's protocol (Qiagen). qPCR was carried out on the StepOnePlus™ (ThermoFisher Scientific) system. Each reaction was set up in technical replicates with wells containing 10 ng total RNA, 10 μM primer pairs, 1×Luna Universal One-Step Reaction Mix, and 1×Luna WarmStart RT Enzyme Mix (Luna® Universal One-Step RT-qPCR Kit, New England Biolabs®). Results were analyzed on the StepOne Software v.2.3 (ThermoFisher Scientific). mRNA expression was calculated using the 2-DDCT method and normalized to the geometric mean of housekeeping genes 18S, POLR2A or GAPDH. Each experiment was repeated at least twice.

### In Vivo Studies

For all in vivo studies 8-10 weeks old female C57BL/6N or C57BL/6J mice were used. To study tumor growth kinetics under ICI and combination treatments, 5×10⁵ CM cells (derived from the spontaneous MT/ret mouse model, BRAF wt, ICI-sensitive), see Helfrich I., et. al., above, or 1.5×10⁶ YUMMER1.7 (BRAF mt, ICI-sensitive), see Wang j., et. al., or 1×10⁵ YUMM1.7 (BRAF mt, ICI-resistant), Meeth, K., et. al., Pigment Cell and Melanoma Research (2016). https://doi.org:10.1111/pcmr.12498, mouse melanoma cells were injected subcutaneously (s.c.) in PBS (YUMM/ER1.7), or in 1:1 mixture of PBS with Matrigel® (CM). The following treatments in different combinations (total injection volume of 200 μl) were administered by intraperitoneal injection: control IgG (IgG2a isotype control clone 2A3, BioXCell, 10 mg/kg body weight, 3x/week) α-CTLA-4 (anti-mouse CTLA-4 clone 9D9, BioXCell, 8 mg/kg body weight, 3x/week), α-PD-1 (anti-mouse PD-1 clone RMP1-14, BioXCell, 10 mg/kg body weight, 3x/week), recombinant mouse IL-17A (IL-17A mouse recombinant, Prospec, 0.01 mg/kg body weight, daily), α-IL-17A (Ultra-LEAF™ purified anti-mouse IL-17A antibody clone TC11-18H10.1, BioLegend, 4 mg/kg body weight, 3x/week), α-Ly6G (anti-mouse Ly6G clone 1A8, Leinco Technologies, 4 mg/kg body weight, 3x/week, starting from day −2) according to the treatment schedule summarized in schematics above corresponding growth curves. Pre-treatment with ICI was done for the CM model. Mice were randomized to different combinatorial treatment groups when tumors became palpable. Treatment continued until tumors had reached the maximal volume (not exceeding 1500 mm³) or became ulcerated. Tumor growth kinetics were analyzed in a long-term experiment, while short-term experiments (end of treatment on day 9 or day 12) were set up to analyze the immune infiltration by multiplex immunofluorescence or flow cytometry and serum cytokine profiles by multiplex cytokine array. Tumor volume was assessed by caliper measurement (calculated as W×W×L/2). At the end of the treatment animals were sacrificed and tumor and blood samples were collected. Tumor samples were fixed in formalin for histological assessment and immunostaining. Blood samples were collected by cardiac puncture in Microvette® 100 Serum tubes (Sarstedt). Serum was separated by standard centrifugation protocol and stored at −80 °C until analysis. Serum samples with significant hemolysis from red blood cells were excluded from cytokine analyses. All animal experiments were performed in accordance with institutional and national guidelines and regulations.

### Multiplex Immunofluorescence

A multiplex immunofluorescence (IF) staining of 4-micron sections, formalin-fixed paraffin-embedded (FFPE) mouse tumor tissues (3 mice/each combination drug treatment group) was executed. De-paraffinization and antigen retrieval was performed using the DAKO PT-Link heat-induced antigen retrieval solution with high pH (pH 9) target retrieval solution (DAKO). Next, each tissue slide was stained in three consecutive rounds of antibody staining, using the Opal Multiplex IHC Kit (Akoya). The slides were washed with Tris-buffered saline containing 0.05% Tween-20® and the microwave treatment was performed in Tris-EDTA buffer (pH 9). If the antibody host species was neither rabbit, nor mouse (as provided in kit), a horseradish peroxidase conjugated secondary antibody for mouse/hamster (Jackson ImmunoResearch) was used at 1:1000 in antibody diluent (Akoya Biosciences), followed by TSA visualization with Opal fluorophores (Akoya Biosciences) diluted in 1×Plus Amplification Diluent (Akoya Biosciences). The IF panels consisted of Melan A (EPR20380, 1:1000, Abcam), Ly6G (RB6-8C5, 1:100, BioLegend), CD8a (C8/144B, 1:100, BioLegend), and Cd11c (N418, 1:100, BioLegend), CD4 (RM4-5, 1:100, BioLegend), IL-17A (TC11-18H10.1, 1:100, BioLegend) primary antibodies. Nuclei were stained by DAPI. Imaging was performed on Zeiss Axio Scan (20× objective) microscopy. The relative contribution of immune cells was calculated by quantitating the background corrected mean fluorescence intensity of each marker at five random fields per tumor tissue and normalized to DAPI. Quantitation was done in ImageJ FIJI software following guidelines by Shihan, M. H., et. al., Biochemistry and Biophysics Reports (2021). https://doi.org:10.1016/j.bbrep.2021.100916.

### Human Patient-Derived Tumor Fragments (PDTFs)

PDTF cultures were performed as previously described, Voabil, P. et al., Nature Medicine (2021). https://doi.org:10.1038/s41591-021-01398-3. In short, tumor specimens were collected from three patients with melanoma undergoing surgery. The tissue was manually dissected into fragments of 1-2 mm³ and cryo-preserved in freezing medium (FCS supplemented with 10% DMSO) until use. Tumor fragments were thawed and embedded in artificial matrix (Cultrex UltiMatrix (Biotechne, 2 mg/mL), rat-tail collagen I (Corning, 1 mg/mL), sodium bicarbonate (Sigma-Aldrich, 1.1%), and DMEM tumor medium (Thermo Fisher Scientific) supplemented with 1 mM of sodium pyruvate (Sigma-Aldrich), 1×MEM nonessential amino acids (Sigma-Aldrich), 2 mM of L-glutamine (Thermo Fisher Scientific), 10% FBS and 1% penicillin-streptomycin) in a 96 well plate, using 8-10 fragments for each treatment condition. For PDTF stimulation, the medium was supplemented with α-PD-1 (10 μg/mL, Nivolumab, Bristol-Myers Squibb), α-CTLA-4 (10 μg/mL, Ipilimumab, Bristol-Myers Squibb) and α-IL-17A (10 μg/mL, clone BL168, Biolegend). After 48 hours of incubation at 37 °C, supernatants were collected, and chemokine and cytokine secretion were assessed using the LEGENDplex Human Th Cytokine and Human Proinflammatory Chemokine assays, according to the manufacturer's protocol.

### Patient Samples

Plasma samples (n=117) from 70 melanoma patients who received first-line ipilimumab plus nivolumab, and plasma samples (n=76) from 51 melanoma patients who received first-line nivolumab or pembrolizumab were collected at therapy baseline and before first staging evaluation (median: week 9, range: 2-12 weeks). All patients were treated at the Department of Dermatology of the University Hospital Essen in standard-of-care or clinical trial settings. Serum samples (n=89) from melanoma patients who received ipilimumab plus nivolumab (n=45) or nivolumab or pembrolizumab (n=44) were collected at therapy baseline across four independent centers (Tübingen, Mannheim, Essen in Germany; St. Gallen in Switzerland). Radiologic tumor response was evaluated by an independent radiologist according to RECIST criteria. Patients with complete response (CR) and partial response (PR) were classified as responders, while with mixed response (MR) and progressive disease (PD) were classified as non-responders. For the Essen cohorts, human biological samples and related data were provided by the Westdeutsche Biobank Essen (WBE/SCABIO, University Hospital Essen, University of Duisburg-Essen, Essen, Germany; approval no. 11-4715, 21-9985-BO). The samples were prospectively collected and archived at the local WBE/SCABIO biobank according to institutional informed consent procedures and retrospectively evaluated for this study. Serum samples in the validation cohorts were collected in compliance with the ethical regulations of the respective institutions' and approval was provided by the Ethical committee of Tübingen University Medical Center (490/2014 B01, 089/2021A), the Ethical committee II of Heidelberg University (2010-318N-MA) and Ethikkommission Ostschweiz (EKOS 16/079). Resected tumor samples were collected from patients with melanoma undergoing surgical treatment at the Netherlands Cancer Institute (NKI-AVL), The Netherlands. The study was approved by the institutional review board of the NKI-AVL (CFMPB484) and executed in compliance with the ethical regulations. All patients consented to the research usage of material not required for diagnostics via prior informed consent.

### Secreted Cytokine Profiling

Secreted levels of human or mouse IL-17A in plasma or serum was determined as per the manufacturer's instructions (LEGEND MAX™ Human IL-17A ELISA Kit, LEGEND MAX™ Mouse IL-17A ELISA Kit, BioLegend). For human samples, plasma samples from psoriasis patients were used as internal reference controls. For multiplex quantification of cytokines, the bead-based LEGENDplex™ panels (Human Th17 7-plex Panel; Human Th 12-plex Panel, Mouse Th17 7-plex Panel; IL-1B, IL-23, IL-12p70 from the Inflammation Panel 1; Granzyme A, Granzyme B from the CD8/NK Panel, pre-defined and custom-designed mix and match system from BioLegend) were used as per the manufacturer's instruction. Flow cytometry reading was performed on the FACSAria™ III (BD). Mean fluorescence intensity (MFI) values were recorded from the LEGENDplex™ analysis software (version 2021. 07.01.) and cytokine concentrations (pg/ml) were interpolated from 5-parameter logistic (5PL) non-linear curve model using separate standard curve for each cytokine. For prognostic stratification of IL-17A plasma levels an optimal cut-point was determined in each dataset separately, using X-tile.

### Results

### The IL-17 Pathway Predicts Clinical Response to Dual ICI

To find a molecular rationale for ICI therapy prediction in melanoma patients based on the observed difference in response to dual ICI between BRAF mt and BRAF wt melanomas, gene expression profiling in a series of treatment-naïve archived tumor samples were performed (discovery set: BRAF mt, V600 hotspot positive melanomas, n=77 and BRAF wt, n=79). To reveal gene expression signatures (GES) in therapeutically relevant immune and resistance pathways, the NanoString™ technology due to its analytical robustness with optimized detection of low-expression RNA targets in archived formalin-fixed paraffin-embedded material was applied. The baseline clinical characteristics of the discovery cohort and details on the NanoString™ gene panels have been recently described see Brase, J. C. et al. Clinical Cancer Research (2021) https://doi.org:10.1158/1078-0432.ccr-20-3586. Differential gene expression analysis revealed diverging transcriptional landscapes between BRAF mt and BRAF wt tumors. There were 21 transcripts significantly upregulated in BRAF mt tumors with enrichment for cytokine- and chemokine-encoding genes (Table 1). In particular, transcriptional signatures indicative of interleukin signaling, especially IL-17, and associated Th17 cell differentiation pathways was found being overrepresented in BRAF mt tumors based on pathway enrichment and gene correlation analyses. In addition, gene set enrichment analysis (GSEA) confirmed IL-17 GES upregulation in BRAF mt tumors.

Since it has been described that IL-17 signaling requires mitogen-activated protein kinase (MAPK) activation, the analyses were expanded to common oncogenic MAPK mutations beyond BRAF V600. Both IL-17 and Th17 cell differentiation GES were among the most significantly overrepresented pathways in MAPK mutated (n=77 BRAF hotspot mt, n=42 NRAS hotspot mt, n=1 NF1 mt) melanomas compared to triple wt melanomas (n=36). To further validate the link between IL-17 signaling GES (defined according to KEGG hsa04657) and the MAPK pathway, data from the largest available melanoma dataset by The Cancer Genome Atlas (TCGA-SKCM) cohort were analysed and found a significant association between IL-17 GES and the MAPK mutational state. Furthermore, this association was also significant when IL-17 GES was correlated with the transcriptional oncogenic activation signature of the MAPK pathway (MAPK-PROGENY). The MAPK pathway plays a role in cellular survival and proliferation, but it is also involved in the production and expression of pro-inflammatory cytokines. Therefore, the oncogenic activation of the MAPK pathway in melanoma cells was correlated to specific cytokines known to regulate IL-17 induction. It has been found that several IL-17-inducing genes were expressed at higher levels in BRAF mt as compared to BRAF wt tumors in the SKCM cohort, and that their expression was significantly decreased in MAPK inhibitor (MAPKi)-treated melanoma tissue biopsies. To further confirm the regulatory axis between MAPK activation and IL-17 regulators, it was demonstrated by pharmacologic manipulation in vitro that IL-17-inducing genes can be expressed by BRAF mt melanoma cells themselves, and dual MAPKi (dabrafenib plus trametinib) leads to decreased transcription of IL-17 regulatory genes.

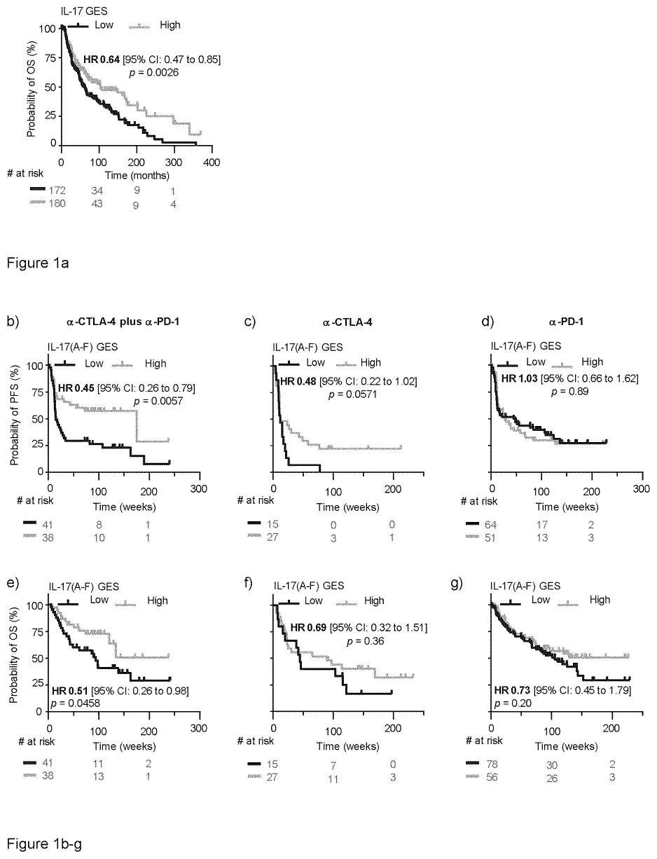

To investigate a potentially relevant prognostic value of baseline IL-17 GES in melanoma tissues that is universal and not necessarily depended only on MAPK signaling, the association between OS and IL-17-signaling in the TCGA-SKCM dataset that consists of mainly untreated melanoma tumors was explored. Indeed, high IL-17 GES was significantly associated with improved OS (HR 0.64, 95% CI 0.47 to 0.85, p=0.0026, FIG. 1a). Then, four different RNA-seq datasets from ICI-treated patient cohorts with metastatic melanoma of various genotypes were analysed (combined cohorts of α-CTLA-4—, α-PD-1-, or α-CTLA-4+α-PD-1, Van Allen et al; Liu et al, Riaz et al, and Gide et al, exact patient numbers are provided in the Methods section). Intriguingly, high expression of core IL-17 signaling genes (‘IL-17A-F GES’: IL-17 family cytokines containing the six structurally related cytokines) predicted longer PFS in dual ICI-treated patients (HR 0.45, 95% CI 0.26 to 0.79, p=0.0057), while it did not correlate with treatment response to mono α-PD-1 or mono α-CTLA-4 therapies (FIG. 1b-d). High IL-17 signaling was also associated with longer OS in dual ICI (HR 0.51, 95% CI 0.26 to 0.98, p=0.0458), but not in mono ICI therapies (FIG. 1e-g).

Overall, these results suggest a co-regulation of the IL-17 and the MAPK pathway, particularly in BRAF mt melanomas where there is strong MAPK activation. However, IL-17 pathway activity is probably not restricted to (known) oncogenic MAPK activators and may instead be a more universal predictor of response to ICI.

### IL-17A is Required for the Anti-Tumor Effect of Dual ICI

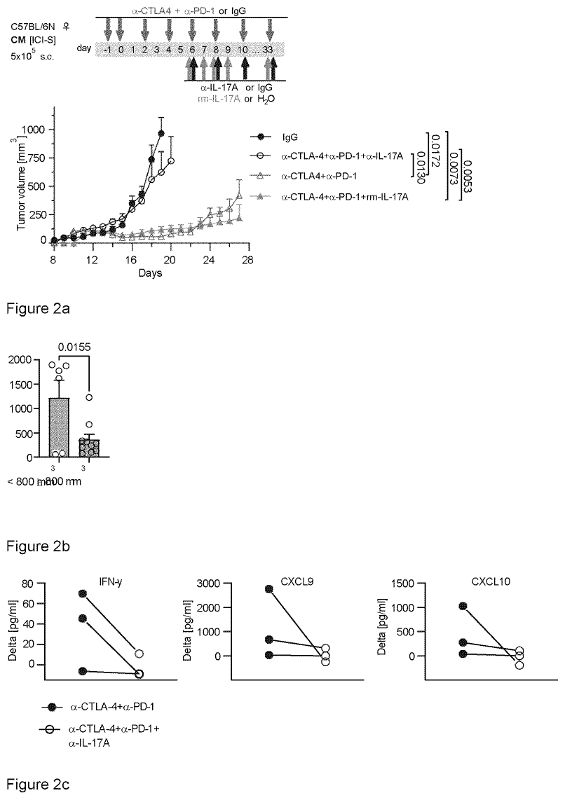

To study the effect of the systemic IL-17A level on the anti-tumor efficacy of ICI therapy in vivo, two syngeneic melanoma transplantation models with distinct genotypes and response profiles to experimentally administered α-CTLA-4 and α-PD-1 antibodies were used, Helfrich et. al., and Qang J. et. al, see above. First, the effects of an IL-17A neutralizing antibody (α-IL-17A) and recombinant mouse IL-17A (rm-IL-17A) on tumor growth kinetics in the ICI-sensitive MT/ret-derived primary cutaneous melanoma (CM) mouse model (human ret transgene, BRAF wt) were examined. As expected, dual ICI significantly slowed down CM tumor growth compared to controls (p=0.0172). Treatment with dual ICI in combination with rm-IL-17A also decreased tumor growth (p=0.0073 vs. controls), whereas the addition of α-IL-17A strongly blocked the anti-tumor effect of dual ICI (p=0.0130 vs. dual ICI, FIG. 2a) and significantly shortened survival. Endpoint analysis of serum IL-17A levels confirmed that the addition of α-IL-17A resulted in significantly less serum IL-17A as compared to levels from dual ICI-treated mice (p=0.0109). Furthermore, a negative correlation between tumor size and serum IL-17A levels was found, with especially big aggressive tumors (≥800 mm³) having significantly lower IL-17A concentrations (p=0.0155, FIG. 2b).

To understand whether IL-17A is also a relevant contributor to CTLA-4+PD-1 blockade in human melanomas, an ex vivo model using patient-derived tumor fragments (PDTFs) was used, which has recently demonstrated high predictive capacity for ICI, see e.g., Voabil P., et. al., PDTFs from three ICI responsive patients' melanomas were treated with dual ICI in the absence or presence of α-IL-17A. In line with the effects observed in the mouse models, α-IL-17A decreased immune activation upon to dual ICI and particularly abrogated IFNγ-induced responses, which are a known as critical driver of clinical responses to ICI (FIG. 2c).

Next, the tumor microenvironment (TME) was characterized to unravel the landscape of IL-17-mediated early immune cell infiltration. A short-term treatment regimen in the CM model (same drug doses) was set up, and performed multiplex immunofluorescence staining. Overall, tumors treated with dual ICI alone or in combination with rm-IL-17A had significantly higher (p<0.05) immune cell infiltration as compared to control. In particular, CD8+ T cells that are the main effectors of therapeutic ICI were increased in tumors treated with dual ICI alone or in combination with rm-IL-17A. Furthermore, CD4+, IL-17A+, CD11c+ cells, and Ly6G+ neutrophils that are potential downstream effectors of IL-17 functions also were significantly enriched in tumors treated with dual ICI alone or in combination with rm-IL-17A, whereas the addition of α-IL-17A counteracted the effect of dual ICI and prevented immune cell infiltration.

Second, it was asked if IL-17 could improve ICI responsiveness also in an intrinsically resistant tumor scenario and applied the YUMM1.7 mouse model, which was reported to lack response to ICI (PTENdel, CDKN2Adel, BRAF V600E mt melanoma). As expected, YUMM1.7 tumors treated with dual ICI showed no response and developed tumors similar to the control (p>0.05 vs. control). However, addition of rm-IL-17A significantly slowed down tumor growth (p=0.0487 vs. control, p=0.0016 vs. dual ICI). Endpoint analysis of serum samples revealed that addition of rm-IL-17A to dual ICI resulted in increased production of IFN-γ, CXCL9 and CXCL10 T cell chemokines, which have been shown to play a role in ICI response and CD8+ T cell recruitment. Together, these findings indicated that increased IL-17 signaling contributes to better response in dual ICI.

### IL-17A/Th17 Cytokines Predict the Response to Dual ICI

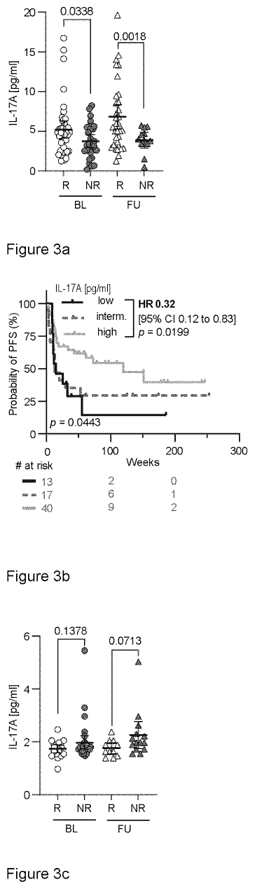

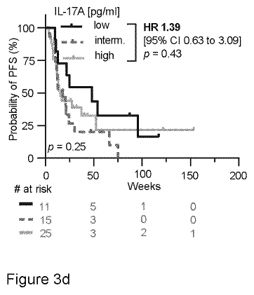

The findings above indicated so far that IL-17 contributes to enhanced dual ICI response and could serve as a therapy stratification biomarker. Therefore, plasma samples of 121 melanoma patients treated at the Essen Department of Dermatology with either first-line dual ICI (α-CTLA-4 plus α-PD-1, n=70) or with first-line α-PD-1 monotherapy (n=51) were analysed. Secreted IL-17A levels in samples collected at therapy baseline (BL) and also at early follow up (FU) visits (median, week 9, range: 2-12 weeks) were significantly higher in therapy responders under dual ICI treatment as compared to non-responders (p=0.0338 at BL, p=0.0018 at FU, FIG. 3a). To test whether BL IL-17A levels could be used as a biomarker for pre-therapeutic therapy stratification, patients were categorized according to their BL IL-17A plasma concentrations. The bioinformatics tool X-tile was applied to achieve the optimal cut-point-based prognostication. It has been found that dual ICI-treated patients with a high BL IL-17A concentration (≥3.76 μg/ml) had longer PFS as compared to patients with intermediate (2.30-3.76 μg/ml, p=0.0682, HR 0.46) or low (≤2.29 μg/ml, p=0.0199, HR 0.32) BL IL-17A (FIG. 3b). To test whether elevated IL-17A is indicative of a global Th17 cytokine profile and phenotype induction, a bead-based multiplex cytokine array including several known Th17, Th1/Th2, inflammatory, and CD8+ T cell/NK (CD8/NK) activation-associated cytokines were applied. Interestingly, dual ICI therapy responders had higher Th17-associated cytokines (IL-10, IFN-γ, IL-17A, and IL-22, p<0.05), particularly at BL. While other inflammatory and CD8/NK cytokines were also elevated in BL and FU samples from responders, they did not statistically stratify patients. In contrast, response to mono α-PD-1 therapy showed no statistically significant correlation with the plasma IL-17A level, although there was a non-significant trend for elevated IL-17 levels in non-responders (FIG. 3c, d). Analysis of additional cytokines in the mono α-PD-1 cohort revealed differences between therapy responders vs. non-responders to a lesser extent, with only baseline IL-6, IL-22, IL-12 (p<0.05) significantly stratifying patients according to response. Interestingly, and in contrast to dual ICI responders, Th17 cytokines were higher in mono α-PD-1 responders in FU, but not in BL plasma samples (statistically non-significant, p=0.5781).

Finally, these findings were validated using a multi-center validation cohort. BL serum samples of 45 melanoma patients treated with dual ICI (α-CTLA-4 plus α-PD-1) and 44 melanoma patients treated with mono α-PD-1 were independently collected across four different Dermatology departments (Tübingen, Mannheim, Essen in Germany; St. Gallen in Switzerland). It has been confirmed that high BL IL-17A levels were associated with dual ICI response (p=0.0401 responders vs. non-responders) and longer PFS (p=0.0230, HR 0.36). In contrast, BL IL-17A levels did not correlate with mono α-PD-1 response (p>0.05).

In conclusion, the data demonstrate that plasma IL-17/Th17 cytokines is a valuable BL biomarker for response prediction and patient stratification in melanoma, specifically to predict a potential benefit of adding α-CTLA-4 to α-PD-1 upfront to therapy.

The role of IL-17 signaling and Th17 cells in cancer progression has been controversially discussed so far, e.g., Ruiz de Morales, J. M. G. et al. in Autoimmunity Reviews (2020). Studies that evaluated the association between IL-17 and patients' prognoses are inconsistent across cancer types including melanoma. Th17 cells and IL-17 are known to have both anti-tumor and pro-tumor effects. However, the underlying mechanism of IL-17 for its anti- or pro-tumor effects in melanoma is not well understood. In mouse models, a few studies supported a pro-tumoral activity of IL-17, where knockdown of IL-17RA or IL-17RC led to decreased formation of B16 melanoma tumors. On the other hand, IL-17A deficient mice have been shown to be susceptible to spontaneous melanoma development 44 or formation of lung tumors. It is described herein that across several published ICI-treated patient cohorts (in total n=79 dual ICI, n=134 mono α-PD-1 and n=42 mono α-CTLA-4 ICI treated patients) that a high baseline IL-17 GES level in melanoma tissue is significantly associated with improved therapy response in dual ICI treated, but not in mono ICI treated patients.

High IL-17 signature expression in ICI treated patient cohorts was additionally positively correlated with higher infiltration of T cells, Th17 cells, dendritic cells, and neutrophils. This suggests that the role of the pre-existent cytokine milieu and that the associated immune cell populations like neutrophils, which are commonly considered a negative predictive marker for ICI, might differ depending on the exact therapeutic ICI context.

IL-17A is the hallmark cytokine of Th17 cells, and is the most potent inducer of downstream cytokines and neutrophil recruitment among IL-17 family members. In brief, it is demonstrated herein that a high baseline IL-17A level in patient plasma samples was indicative of a higher global baseline Th17 cytokine profile preceding clinical response to dual ICI in the metastatic setting, but not mono α-PD-1. In the ipilimumab dose-ranging study BRAF mt patients had longer median OS as compared to BRAF wt patients with the high 10 mg/kg but also the standard 3 mg/kg dose of ipilimumab (33.2 vs. 8 months and 19.7 vs. 2 months, respectively), Ascierto, P. A. et al., Journal for Immuno Therapy of Cancer 8, e000391-e000391 (2020). https://doi.org:10.1136/jitc-2019-000391. This could indicate that actually ipilimumab is a drug that is predominantly IL-17 responsive also when given as combination in dual ICI. Furthermore, the association between IL-17 and MAPK activation points to further biomarker opportunities for triple combination (MAPKi+ICI) therapies.

In sum, the data show that IL-17 is suitable as a biomarker for predicting response to dual ICI therapy. IL-17 cytokine levels can be measured by common analytical biochemistry assays (e.g., ELISA) that are easily accessible and applicable in the clinical routine across institutions.

1. A method of determining (i) whether a subject who has been diagnosed with melanoma is amenable to a treatment with one or more immune checkpoint inhibitors (ICI)(s), (ii) a therapy regimen of a subject who as been diagnosed with melanoma whereby the therapy regimen comprises treatment with one or more ICI(s), or (iii) responsiveness of a subject who has been diagnosed with melanoma to a treatment with one or more ICI(s), comprising:
a) measuring a level and/or an amount of IL-17 in a biological sample obtained from the subject;
b) drawing a conclusion as to (i) the amenability of said subject to a treatment with ICI(s), (ii) the therapy regimen of said subject to a treatment with ICI(s), or (iii) the extent of responsiveness of said subject to a treatment with ICI(s) from the level and/or amount of IL-17 measured in step a).

2. (canceled)

3. (canceled)

4. The method according to claim 1 wherein the ICI(s) comprises at least one of an anti-PD-1 ICI and/or an anti-CTLA-4 ICI.

5. The method according to claim 1 wherein the melanoma is a metastatic melanoma.

6. The method according to claim 1 wherein the biological sample is tissue or blood.

7. The method according to claim 1 wherein the IL-17 measured is IL-17a.

8. The method according to claim 1 wherein elevated levels of IL-17 measured in step a) is indicative for beneficial treatment with a combinatorial therapy of at least two different ICI(s), whereas when the IL-17 level or amount is not increased, monotherapy with a single ICI selected from an anti-PD-1 ICI or anti-CTLA-4 ICI is suitable, optionally the treatment or therapy is combined with an administration of IL-17.

9. The method according to claim 8 wherein the subject having elevated levels of IL-17 will have an increased survival rate when treated with a combination of two different ICI(s), optionally the treatment of therapy is combined with an administration of IL-17.

10. (canceled)

11. (canceled)

12. (canceled)

13. A pharmaceutical composition comprising a combination of IL-17, at least one of anti-PD-1 ICI and anti-CTLA-4 ICI.

14. (canceled)

15. (canceled)

16. A method for treating melanoma in a subject in need thereof, comprising:
i) measuring a level and/or an amount of IL-17 in a biological sample obtained from the subject;
ii) administering a pharmaceutical composition containing one or more immune checkpoint inhibitors (ICI)(s) and IL-17 to the subject different components of the pharmaceutical composition may be administered simultaneously, separately or sequentially.

17. (canceled)

18. The method according to claim 16 wherein the melanoma is metastatic melanoma.

19. The method of claim 16 wherein based on a measurement that the subject has an elevated level or amount of IL-17, the administering step provides the subject with at least two different ICIs.

20. The method of claim 19 wherein the two different ICIs are anti-PD-1 ICI and anti-CTLA-4 ICI.

21. The method of claim 16 wherein based on a measurement that the subject does not have an elevated level or amount of IL-17, the administering step provides the subject with only a single ICI.

22. The method of claim 21 wherein the single ICI is selected from the group consisting of anti-PD-1 ICI and anti-CTLA-4 ICI.

23. The method of claim 16 wherein a) administration of IL-17 is followed by ICI administration (mono or combination) or b) administration of ICI (mono or combination) followed by IL-17 or c) administration of IL-17/ICI(s) in the same at the same time or d) administration of IL-17 followed by ICI mono or combination with different time intervals in between in infusions whereby IL-17 is administered intratumorally or peritumorally while ICI is administered i.v. or e) administration of ICI(s) (mono or combination) i.v. followed by IL-17 peritumorally or intratumorally or f) administration of ICI(s) (mono or combination) i.v. and IL-17 peritumorally or intratumorally at the same time.
